(19) United States
(12) Patent Application Publication
(10) Pub. No.: US 2025/0366896 A1
(43) Pub. Date: Dec. 4, 2025

(54) Method for reducing a risk of a potential fracture in an osteoporotic vertebra

(71) Applicant: JOY MEDICAL DEVICES CORPORATION, Kaohsiung (TW)

(72) Inventors: Chien-Ping JU, Kansas City, MO (US); Jiin-Huey Chern LIN, Winnetka, IL (US)

(21) Appl. No.: 18/731,565

(22) Filed: Jun. 3, 2024

(51) Int. Cl. A61B 17/88; A61B 17/00; A61B 17/56

(52) U.S. Cl. CPC A61B 17/8816; A61B 2017/00004; A61B 2017/564

(57) ABSTRACT

A percutaneous minimally-invasive procedure to prevent a potential fracture in an osteoporotic vertebra of an osteoporotic patient is provided by minimally-invasively injecting a non-dispersive, biocompatible and resorbable calcium-based cement paste into the osteoporotic vertebral body.

### FIELD OF THE INVENTION

The present invention is related to a minimally invasive method to prevent a potential fracture in an osteoporotic vertebra of an osteoporotic patient who has recovered from a treatment of vertebral fracture.

### BACKGROUND OF THE INVENTION

U.S. Pat. No. 11,399,960 B2 discloses a method for reducing a risk of a potential fracture in an osteoporotic vertebra of an osteoporotic patient comprising identifying an osteoporotic patient who has no history of vertebral fracture, and identifying a vertebra suffering osteoporosis, wherein said osteoporotic vertebra is diagnosed with a T-score lower than −2.5; injecting a calcium-based cement paste into said osteoporotic vertebra, wherein the injected calcium-based cement paste will harden in said osteoporotic vertebra to form a hardened block, and at least a portion of the hardened block is bioresorbable and will be gradually replaced by a newly-grown bone structure in said osteoporotic vertebra, wherein said injecting said calcium-based cement paste into said osteoporotic vertebra comprises drilling a hole on said osteoporotic vertebra, followed by inserting a distal end of a thin tube into said hole so that an empty space created directly by said drilling in said osteoporotic vertebra is fluidly communicated with a proximal end of said thin tube, and feeding said thin tube with said calcium-based cement paste from said proximal end thereof until a desired amount of said calcium-based cement paste leaves said distal end of the thin tube and enters said empty space in said osteoporotic vertebra.

### SUMMARY OF THE INVENTION

The present invention is related to a minimally invasive method to prevent a potential fracture in an osteoporotic vertebra of an osteoporotic patient who has a history of vertebral fracture and has recovered from a treatment of the vertebral fracture or who has no history of vertebral fracture and has received a treatment of reducing a risk of vertebral fracture by injecting a calcium-based cement paste into said osteoporotic vertebra, which comprises first identifying an osteoporotic vertebra in said patient;
injecting a calcium-based cement paste into said osteoporotic vertebra, wherein the injected calcium-based cement paste will harden in said osteoporotic vertebra to form a hardened block, and at least a portion of the hardened block is bioresorbable and will be gradually replaced by a newly-grown bone structure in said osteoporotic vertebra.

Preferably, said injecting the calcium-based cement paste into said osteoporotic vertebra comprises drilling a hole on said osteoporotic vertebra, creating a cavity in said osteoporotic through said hole, inserting a distal end of a thin tube into said hole so that the cavity is fluidly communicated with a proximal end of said thin tube, and feeding said thin tube with said calcium-based cement paste from said proximal end thereof until a desired amount of said calcium-based cement paste leaves said distal end of the thin tube and enters said cavity.

Preferably, said injecting the calcium-based cement paste into said osteoporotic vertebra comprises drilling a hole on said osteoporotic vertebra, inserting a distal end of a thin tube into said hole so that an empty space in said osteoporotic vertebra is fluidly communicated with a proximal end of said thin tube, and feeding said thin tube with said calcium-based cement paste from said proximal end thereof until a desired amount of said calcium-based cement paste leaves said distal end of the thin tube and enters said empty space in said osteoporotic vertebra.

### DETAILED DESCRIPTION OF THE INVENTION

It is the present Inventors' belief that for those who have recovered from a treatment of a vertebral fracture or for those who have no histories of vertebral fracture and have received a treatment of reducing a risk of a potential fracture in an osteoporotic vertebra as described in the aforesaid U.S. Pat. No. 11,399,960 B2, there is a relatively higher risk of suffering an osteoporotic vertebra again in view of their histories of vertebral fracture or having an osteoporotic vertebra.

In the present invention, the method disclosed in U.S. Pat. No. 11,399,960 B2 is applied to prevent a potential fracture in an osteoporotic vertebra of an osteoporotic patient who has a history of vertebral fracture and has recovered from a treatment of the vertebral fracture or who has no history of vertebral fracture and has received a treatment of reducing a risk of vertebral fracture. The disclosure in U.S. Pat. No. 11,399,960 B2 is incorporated herein by reference.

1. A method for reducing a risk of a potential fracture in an osteoporotic vertebra of an osteoporotic patient comprising:
identifying an osteoporotic patient from those who have recovered from a treatment of vertebral fracture, which comprises identifying a vertebra suffering osteoporosis in said osteoporotic patient, wherein said osteoporotic vertebra is diagnosed with a T-score lower than −2.5; and
injecting a calcium-based cement paste into said osteoporotic vertebra, wherein the injected calcium-based cement paste will harden in said osteoporotic vertebra to form a hardened block, and at least a portion of the hardened block is bioresorbable and will be gradually replaced by a newly-grown bone structure in said osteoporotic vertebra.

2. The method of claim 1, wherein said injecting said calcium-based cement paste into said osteoporotic vertebra comprises drilling a hole on said osteoporotic vertebra, followed by inserting a distal end of a thin tube into said hole so that an empty space created directly by said drilling in said osteoporotic vertebra is fluidly communicated with a proximal end of said thin tube, and feeding said thin tube with said calcium-based cement paste from said proximal end thereof until a desired amount of said calcium-based cement paste leaves said distal end of the thin tube and enters said empty space in said osteoporotic vertebra.

3. A method for reducing a risk of a potential fracture in an osteoporotic vertebra of an osteoporotic patient comprising:
identifying an osteoporotic patient from those who have received a treatment of reducing a risk of a potential fracture in an osteoporotic vertebra by injecting a bone cement into said osteoporotic vertebra, which comprises identifying a vertebra suffering osteoporosis in said osteoporotic patient, wherein said osteoporotic vertebra is diagnosed with a T-score lower than −2.5; and
injecting a calcium-based cement paste into said osteoporotic vertebra, wherein the injected calcium-based cement paste will harden in said osteoporotic vertebra to form a hardened block, and at least a portion of the hardened block is bioresorbable and will be gradually replaced by a newly-grown bone structure in said osteoporotic vertebra.

4. The method of claim 3, wherein said injecting said calcium-based cement paste into said osteoporotic vertebra comprises drilling a hole on said osteoporotic vertebra, followed by inserting a distal end of a thin tube into said hole so that an empty space created directly by said drilling in said osteoporotic vertebra is fluidly communicated with a proximal end of said thin tube, and feeding said thin tube with said calcium-based cement paste from said proximal end thereof until a desired amount of said calcium-based cement paste leaves said distal end of the thin tube and enters said empty space in said osteoporotic vertebra.
